(19) United States
(12) Patent Application Publication
(10) Pub. No.: US 2005/0112059 A1
(43) Pub. Date: May 26, 2005

(54) Enhancement of radiotherapy by an exogenous cardiac glycoside

(75) Inventors: Robert A. Newman, Houston, TX (US); Crandell Addington, San Antonio, TX (US); Sachiko Nasu, Tunina (JP); Luka Milas, Houston, TX (US); Shinichiro Kawabe, Osaka (JP); Uma Raju, Missouri City, TX (US)

Correspondence Address: JACKSON WALKER LLP, 2435 NORTH CENTRAL EXPRESSWAY, SUITE 600, RICHARDSON, TX, 75080, US

(73) Assignees: Phoenix Biotechnology, Inc., San Antonio, TX (US); The University of Texas, Houston, TX (US)

(21) Appl. No.: 10/957,875

(22) Filed: Oct. 4, 2004

Related U.S. Application Data
(60) Provisional application No. 60/508,936, filed on Oct. 6, 2003.

(51) Int. Cl. A61K 51/00; A61K 31/704

(52) U.S. Cl. 424/1.11; 514/26; 600/1

(57) ABSTRACT

A method for enhancing the radiosensitivity of cells through the administration of an exogenous cardiac glycoside, such as oleandrin. The magnitude of radiosensitization depends on the duration of exposure of the cells to oleandrin prior to irradiation. Treatment of cells with oleandrin increases the sensitivity of the cells to radiation-induced apoptosis. Thus, treatment with oleandrin and radiotherapy effectively lessens the proliferation of tumor cell populations.

### BACKGROUND

This application claims priority to U.S. Provisional Patent Application Ser. No. 60/508,936, entitled “Enhancement of Radiotherapy by an Exogenous Cardiac Glycoside” filed on Oct. 2, 2003, the entire content of which is hereby incorporated by reference.

This invention pertains to the use of an exogenous cardiac glycoside, such as oleandrin, to enhance the radiosensitivity of a population of tumor cells. The current invention also pertains to the use of an exogenous cardiac glycoside, in conjunction with radiotherapy, to treat cancer patients.

Radiotherapy is an integral weapon in the fight against cancer. It is certain to become even more so as today's technology develops ever more precise and effective treatment. When dealing with any radiation, the question of causing or promoting new cancers always arises. High doses of radiation can produce cancer in time. In using radiation therapy or any other treatment, one must always weigh the risks against the benefits. The potential benefit of using radiation therapy to cure a cancer obviously outweighs any risk of possibly causing another cancer at some future date. Nevertheless, it is a goal of radiotherapy to use as little, but effective, doses of radiation as possible.

Cardiac glycosides are a class of natural products that have been traditionally used to increase cardiac contractile force in patients suffering from congestive heart failure. These agents have the ability to inhibit Na⁺, K⁺-ATPase, which results in modification of Na⁺, K⁺ and Ca⁺⁺ ion fluxes in cells. Specifically, cardiac glycosides maintain elevated intracellular K⁺ and a decreased intracellular Na⁺, compared to extracellular fluid. There are, however, suggestions that cardiac glycosides may also play a role in the treatment of cancer (Stenkvist et al., Lancet, vol. 1, p. 563, 1979; Stenkvist, Oncol. Rep., vol. 6, pp. 493-496, 1999). A recent publication, noted that breast carcinoma patients who were on digitalis medication at the time of cancer diagnosis had significantly better response to anticancer therapy and better overall survival than breast cancer patients who were not taking digitalis (Stenkvist, Oncol. Rep., vol. 6, pp. 493-496, 1999). The antitumor efficacy exhibited by the glycosides is supported by studies showing that these agents can be selectively cytotoxic to tumor cells in vitro (Haux, Med. Hypotheses, vol. 53, pp. 543-548, 1999). Among these agents is an extract from Nerium oleander.

The extract contains oleandrin, a cardiac glycoside that is exogeneous and not normally present in the body, whose structure is similar to that of other cardiac glycosides. Oleandrin induces apoptosis in human but not in murine tumor cell lines (Pathak et al., Anti-Cancer Drugs, vol. 11, pp. 455-463, 2000), inhibits activation of NF-kB (Manna et al., Cancer Res., vol. 60, pp. 3838-3847, 2000), and mediates cell death through a calcium-mediated release of cytochrome C (McConkey et al., Cancer Res., vol. 60, pp. 3807-3812, 2000). A Phase I trial of an oleander extract has been completed recently (Mekhail et al., Am. Soc. Clin. Oncol., vol. 20, p. 82b, 2001). It was concluded that oleander extracts can be safely administered at doses up to 1.2 ml/m²/d. No dose limiting toxicities were found.

In addition to being selectively cytotoxic for tumor cells, cardiac glycosides may also enhance cell response to cytotoxic actions of ionizing radiation. Ouabain, a cardiac glycoside endogeneous to the body, was reported to enhance in vitro radiosensitivity of A549 human lung adenocarcinoma cells but was ineffective in modifying the radioresponse of normal human lung fibroblasts (Lawrence, Int. J. Radiat. Oncol. Biol. Phys., vol. 15, pp. 953-958,1988). Ouabain was subsequently shown to radiosensitize human tumor cells of different histology types including squamous cell carcinoma and melanoma (Verheye-Dua et al., Strahlenther. Onkol., vol. 176, pp. 186-191, 2000). Although the mechanisms of ouabain-induced radiosensitization are still not fully explained, inhibition of repair from sublethal radiation damage and an increase in radiation-induced apoptosis have been advanced as possibilities (Lawrence, 2000; Verheye Dua et al., 2000; Verheye-Dua et al., Strahlenther. Onkol., vol. 172, pp. 156-161, 1996).

U.S. Pat. No. 5,135,745 to Ozel pertains to extracts of Nerium species and their use in the treatment of cell-proliferative diseases. The effects of treatment upon sensitivity to radiotherapy were not explored, although it was noted that pretreatment with chemotherapy or radiotherapy had a tendency to lessen the positive effects of the Nerium treatment.

U.S. Pat. No. 5,869,060 to Yoon et al. pertains to the use of extracts of Portulaca oleracea, a plant containing a cardiac glycoside, to inhibit tumor cell growth. The extracts were not used in combination with chemotherapy or radiotherapy.

U.S. Pat. No. 5,872,103 to Belletti pertains to the prevention of mammary tumors through treatment with cardiac glycosides. The cardiac glycosides were shown to have a prophylactic activity through preventing the development of neoplasia or cancer in individuals identified as having a high risk of developing these conditions.

U.S. Pat. No. 6,380,167 to Braude pertains to the use of digitoxin, acardiac glycoside, to treat tumor cells. Effects on sensitivity to chemotherapy or radiotherapy were not investigated.

U.S. Pat. No. 6,565,897 to Selvaraj et al. pertains to the use of extracts of the Nerium species to treat cell-proliferative diseases. Post-treatment effects on the sensitivity of the tumor cells to chemotherapy and radiotherapy was not explored.

It is clear that there is a need to have safer cancer cures through radiotherapy. The goal of radiotherapy should be to use as little, but still effective, doses of radiation as possible.

### SUMMARY

The present invention relates to a method for enhancing the radiosensitivity of a population of cells, such as tumor cells. Thus, the amount, or doses, of radiation needed to achieve the same kill of cells, or tumor cells, can be reduced when the radiosensitivity of cells, or tumor cells, is enhanced. The method comprises treating the cell population, such as a population of tumor cells, with a predetermined radiosensitivity-enhancing amount of an cardiac glycoside, such as oleandrin. The cardiac glycoside, such as oleandrin, can be an exogenous cardiac glycoside. The predetermined radiosensitivity-enhancing amount of oleandrin is an amount needed to enhance the radiosensitivity of the cell population, such as a tumor cell population. The present invention also relates to a method for lessening proliferation of a cell population comprising treating the cell population to a radiosensitivity-enhancing amount of oleandrin to give a treated cell population, and then exposing this treated cell population to an effective amount of ionizing radiation. The cell population can be a tumor cell population.

The exogenous cardiac glycoside oleandrin has the ability to enhance the sensitivity of cells to the cytotoxic action of ionizing radiation. Thus, the present invention also relates to a method for lessening the proliferation of cell populations and a method for treating tumors, through the administration of oleandrin together with radiation. The magnitude of radiosensitization depends on the duration of exposure of the cells to oleandrin prior to irradiation. Treatment of cells with oleandrin increases the sensitivity of the cells to radiation-induced apoptosis. Thus, treatment with oleandrin and radiotherapy effectively lessens the proliferation of tumor cell populations.

### BRIEF DESCRIPTION OF THE DRAWINGS

FIG. 1 shows the effect of oleandrin on radiosensitivity of PC-3 cells in culture.

FIG. 2 shows time dependent cytotoxic effect of oleandrin on the PC-3 cells.

FIG. 3 shows induction of apoptosis by oleandrin in PC-3 cells.

FIG. 4 shows expression of procaspase-3 and capase 3 proteins by Western blot analysis.

### DETAILED DESCRIPTION OF PREFERRED EMBODIMENTS

The present invention relates to a method for enhancing the radiosensitivity of a population of cells. This cell population may be tumor cell population. Thus, the amount, or doses, of radiation needed to achieve the same kill of cells, such as tumor cells, can be reduced when the radiosensitivity of cells, such as tumor cells, is enhanced. The method comprises treating the cell population, such as a population of tumor cells, with a predetermined radiosensitivity-enhancing amount of a cardiac glycoside, such as oleandrin. The cardiac glycoside used in the present invention can be any established therapeutic compound known as a cardiac glycoside. The exogenous cardiac glycoside denotes the cardiac glycoside that is produced or originated from outside the body, such as the body of a patient. Other cardiac glycosides include, for example, digoxin, digitoxin, ouabain, and bufalin. The radiosensitivity-enhancing amount is the amount need to enhance the radiosensitivity of the cell population. Depending on the type of cells, the concentration amount can vary from about 0.01 μg/mL to about 2 μg/mL. The present invention also relates to a method for lessening the proliferation of a given cell population, such as a population of tumor cells. The method comprises treating the cell population with a predetermined radiosensitivity-enhancing amount of a cardiac glycoside, such as exogenous oleandrin, and then exposing this treated cell population to an effective amount of ionizing radiation. The cell population can be a tumor cell population. Again, the amount of ionizing radiation will vary depending on the types of cells.

Oleandrin, in addition to being cytotoxic to PC-3 human prostate carcinoma cells, has the ability to enhance the sensitivity of these cells to cytotoxic action by ionizing radiation. After the administration of oleandrin, cells are exposed to an amount of radiation effective to decrease the cell population. Radiosensitivity is observed over a range of doses of radiation, and the enhancement of radiosensitivity is similar to enhancement values reported for ouabain. The amount of radiosensitization of the cells depends on the duration of the cells' exposure to oleandrin. Radiosensitization occurs after incubation of cells with oleandrin for 1 hour, but the amount of radiosensitization increases with an increase in incubation time. Oleandrin enhances the radioresponse of tumor cells by rendering the cells more sensitive to radiation induced apoptosis. The method of the present invention appears to be of significant use for the enhancement of radiosensitivity of prostate carcinoma cells and the lessening of their proliferation.

At the molecular level, the susceptibility of PC-3 cells to oleandrin and radiation-induced apoptosis is dependent on the activation of the protein caspase-3. The inhibition of caspase-3 abrogates the radiosensitizing ability of oleandrin. Treatment with radiation alone does not induce any measurable amount of activated caspase-3. However, co-treatment of cells with both oleandrin and radiation produces an even greater formation of caspase-3, indicating that oleandrin made PC-3 cells susceptible to radiation-induced activation of caspase-3.

### EXAMPLE 1

### Effect of Oleandrin on Radiosensitivity

The human prostate carcinoma cell line PC-3 was obtained from the American Type Culture Collection and maintained in RPMI 1640 medium supplemented with 10% fetal calf serum, 10,000 U/ml of penicillin-streptomycin, and 2 mM L-glutamine. Cells were grown as monolayers in 75-cm² flasks and maintained in a humidified 5% CO₂/95% air atmosphere at 37.8° C.

The PC-3 cells were exposed to 0.05 μg/ml oleandrin for 24 hours and then irradiated with graded doses (2, 4 or 6 Gy) of γ-rays using a ¹³⁷Cs source (3.7 Gy/min) and assayed for colony forming ability by replating them in specified numbers into 100-mm dishes containing drug-free media. After 12 days of incubation, cells were stained with 0.5% crystal violet in absolute ethanol, and colonies with more than 50 cells were counted. The average survival levels were fit by least squares regression using a linear quadratic model. Survival curves were constructed with normalized values for the cytotoxicity induced by oleandrin alone. Oleandrin by itself reduced plating efficiency from the 95% control value to 21%.

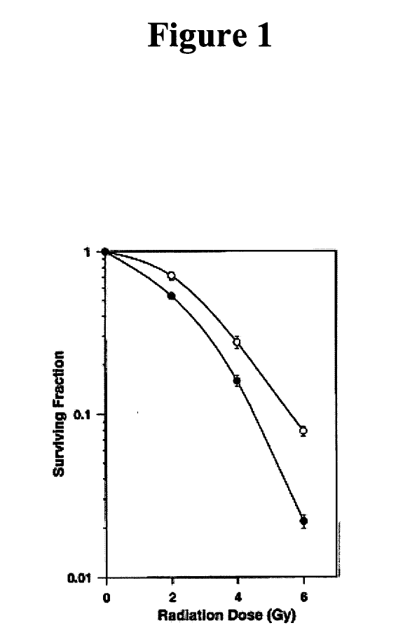

As shown in FIG. 1, treatment with oleandrin resulted in an increased response of PC-3 cells to radiation. The untreated control samples are represented by the open circles and the oleandrin treated samples are represented by the filled circles. Values shown are the means±SE for three independent experiments. The enhancement factor at the cell survival fraction of 0.1 was 1.32. The concentration of oleandrin at 0.025 μg/ml, which by itself was non-cytotoxic, had no significant influence on radiosensitization of PC-3 cells and the data are not presented.

### EXAMPLE 2

### Effect of Duration of Oleandrin Exposure on Radiosensitivity

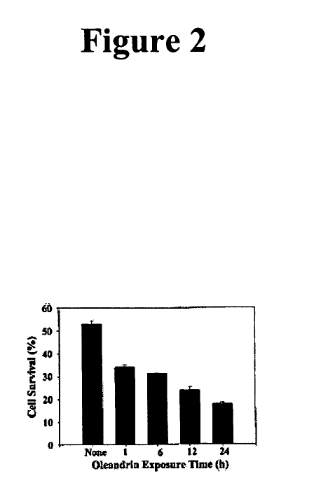

The magnitude of radiosensitization depended on duration of exposure of cells to oleandrin prior to radiation. PC-3 cells were incubated in the presence of 0.05 μg/ml oleandrin for 1-24 hours. After oleandrin treatment, cells were irradiated with 3 Gy radiation and plated as in Example 1. Colonies were counted after 12 days and percent surviving fractions were plotted. As shown in FIG. 2, cell exposure to oleandrin for only 1 hour enhanced radiation-induced cell death. Cell survival after 3 Gy radiation only was 50% compared to 30% after 3 Gy radiation plus 0.05 μg/ml oleandrin. Values shown are means±SE for three independent experiments. The average survival levels were fit by least squares regression using a linear quadratic model. The radiosensitizing effect of oleandrin increased as the duration of exposure to oleandrin increased up to 24 hours.

### EXAMPLE 3

### Effects of Oleandrin on Susceptibility of Cells to Radiation—Induced Apoptosis

PC-3 cells were incubated with 0.05 μg/ml oleandrin for 24 hours, irradiated at 2 Gy, or both. The control cells were untreated. The medium containing oleandrin was removed, fresh medium was added, and 24 hours later the apoptotic index was determined.

Apoptosis was determined by staining and flow cytometry analysis. The terminal deoxynucleotidyltransferase (“TdT”) dUTP nick end labeling (“TUNEL”) assay to identify the DNA fragmentation (APO-DIRECT kit, Pharmingen, San Diego, Calif.) was performed according to the manufacturer's instructions. Briefly, cells (2×10⁶) were fixed in 1% paraformaldehyde and washed in PBS. Cells were suspended in 70% ethanol and stored at −20° C. until use. Re-suspended cells were stained in a solution containing TdT and FITC-dUTP and incubated overnight at room temperature in the dark. They were then rinsed and re-suspended in 0.5 ml propidium iodide/RNase A solution and analyzed by flow cytometry.

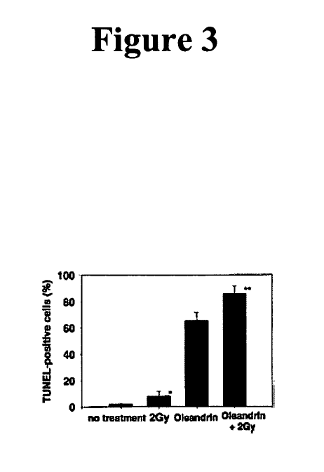

FIG. 3 shows the induction of apoptosis by oleandrin in PC-3 cells. The data shown are mean±SE for three independent experiments. FIG. 3 shows that 2 Gy increased the percentage of apoptotic cells from the control value of 2.1±0.4 to 8.2±3.4%, oleandrin increased it to 63.5±6.1%, and the combination of oleandrin and 2 Gy increased it to 85.8±5.3%. The latter was more than the additive effects of oleandrin and 2 Gy when given as individual treatments.

In addition, the control cells and the experimental cells were stained with a fluorescent dye, Hoechst 33258, that binds to fragmented DNA. The PC-3 cells were observed under a fluorescent microscope. More apoptotic cells were observed in the oleandrin plus radiation group than in any other treated or control cell population.

### EXAMPLE 4

### Evidence of Apoptosis in Expression of Procaspase-3 and Caspase-3 proteins

To determine whether caspase-3 activation was involved in the oleandrin-induced enhancement of PC-3 cell radioresponse, cells were treated with 5 Gy or 0.05 μg/ml oleandrin or a combination of both treatments for 24 hours. Cells were assayed for caspase-3 activation 24 hours after treatment using western blotting. cells were lysed in a buffer containing 50 mM Tris-HCl (pH 8), 450 mM NaCl, 1% triton X-100, 5 mM EDTA, 1% (v/v) of protease inhibitor cocktail, and 1% (v/v) phosphatase inhibitor cocktails I and II (Sigma, St. Louis, Mo.). Protein (30-40 mg per lane) was separated by sodium dodecyl sulfate-polyacrylamide gel electrophoresis (“SDS-PAGE”) and transferred to a polyvinylidene difluoride (“PVDF”) membrane (Bio-Rad Laboratories, Hercules, Calif.). The membrane was blocked by 5% non-fat dry milk in Tris-buffered saline and 0.1% Tween-20 (“TBS-T”) and before incubation with a designated primary antibody (Pharmingen, San Diego, Calif.). After washing the membrane with TBS-T, it was incubated in secondary antibody and the immunoreaction was visualized using an ECL western blotting kit (Amersham Corp., Arlington Heights, Ill.).

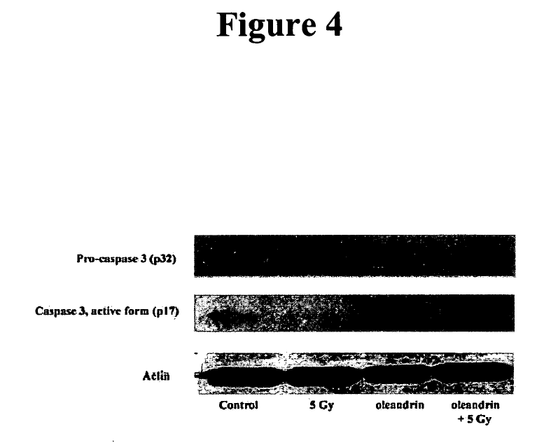

As shown in FIG. 4, radiation alone was ineffective, whereas oleandrin was effective in activating caspase-3. However, the level of activation was higher when the two agents were combined, indicating that oleandrin made PC-3 cells susceptible to radiation-induced activation of caspase-3.

### EXAMPLE 5

### Effects of Caspase-3 Inhibitors on Enhancement of Radiosensitivity

PC-3 cells were incubated with 0.05 μg/ml oleandrin for 24 hours before being irradiated with 2 or 6 Gy. ZDEVD-FMK, an inhibitor of caspase-3, at a dose of 100 μM was added 2 hours before oleandrin. Clonogenic cell survival was then determined. Colonies were counted and the survival of cells (percent control) was calculated. Results are shown in Table 1. Data are presented as mean±SE from three experiments.

TABLE 1
Effect of caspase-3 inhibitor on oleandrin-induced radiosensitivity

| | Survival of cells (%) | | |
|---|---|---|---|
| | following radiation of | | |
| Treatment | O Gy | 2 Gy | 6 Gy |
| Control | 100 | 74.6 ± 3.4 | 7.8 ± 0.6 |
| Oleandrin | 88.9 ± 2.9 | 55.5 ± 1.9 | 3.1 ± 0.8 |
| Z-DEVD-FMK | 97.4 ± 1.7 | 77.8 ± 3.2 | 8.6 ± 2.1 |
| Z-DEXVD-FMK + Oleandrin | 104 ± 1.6 | 70.4 ± 2.2 | 62 ± 0.8 |

The results in Table 1 show that the inhibitor of caspase-3 (ZDEVD-FMK) abolished the oleandrin-induced enhancement of radiation response. However, the inhibitor alone did not significantly affect the radioresponse of PC-3 cells.

### REFERENCES CITED

The following U.S. patent documents are hereby incorporated by reference.

### U.S. Patent Documents

U.S. Pat. No. 5,135,745 to Ozel
U.S. Pat. No. 5,869,060 to Yoon et al.
U.S. Pat. No. 5,872,103 to Belletti
U.S. Pat. No. 6,380,167 to Braude
U.S. Pat. No. 6,565,897 to Selvaraj et al.

### Other Publications

Haux, J., Digitoxin is a potential anticancer agent for several types of cancer, Med. Hypotheses vol. 53 (1999) 543-548.
Lawrence, T. S., Ouabain sensitizes tumor cells but not normal cells to radiation, Int. J. Radiat. Oncol. Biol. Phys. vol. 15 (1988) 953-958.
Manna, S. K., N. K. Sah, R. A. Newman, A. Cisneros, B. B. Aggarwal, Oleandrin suppresses activation of nuclear transcription factor-kB, activator protein-1, and c-jun N-terminal kinase, Cancer Res. vol. 60 (2000) 3838-3847.
McConkey, D. J., Y. Lin, K. Nutt, H. Z. Ozel, R. A. Newman, Cardiac glycosides stimulate calcium increases and apoptosis in androgen-dependent metastatic human prostate adenocarcinoma cells, Cancer Res. vol. 60 (2000) 3807-3812.
Mekhail, T., C. Kellackey, T. Hutson, T. Olencki, G. T. Budd, D. Peereboom, R. Dreicer, P. Elson, R. Ganaphthi, R. Bukowski, Phase I study of Anvirzel in patients with advanced solid tumors, Am. Soc. Clin. Oncol. vol. 20 (2001) 82b.
Pathak, S., A. S. Multani, S. Narayan, V. Kumar, R. A. Newman, Anvirzel: an extract of Nerium oleander induces cell death in human but not murine cancer cells, Anti-Cancer Drugs vol. 11 (2000) 455-463.
Stenkvist, B., E. Pengtsson, O. Eriksson, J. Holmqvist, B. Nordin, S. Westman-Naeser, Cardiac glycosides and breast cancer, Lancet vol. 1 (1979) 563.
Stenkvist, B., Is digitalis a therapy for breast carcinoma?, Oncol. Rep. 6 (1999) 493-496.
Verheye-Dua, F. A., L. Bohm, Influence of cell inactivation by irradiation, Strahlenther. Onkol. vol. 172 (1996) 156-161.
Verheye-Dua, F. A., L. Bohm, Influence of apoptosis on the enhancement of radiotoxicity by ouabain, Strahlenther. Onkol. vol. 176 (2000) 186-191.

1. A method for enhancing radiosensitivity of a cell population comprising:
exposing the cell population to a predetermined amount of an exogenous cardiac glycoside.

2. The method of claim 1, wherein the cell population comprises tumor cells.

3. The method of claim 1, wherein the exogenous cardiac glycoside comprises oleandrin, ouabain, digoxin, digitoxin, or bufalin.

4. A method for lessening proliferation of a cell population comprising:
treating the cell population with a radiosensitivity-enhancing amount of an exogenous cardiac glycoside to give a treated cell population; and
exposing the treated cell population to an effective amount of ionizing radiation.

5. The method of claim 4, wherein the cell population comprises tumor cells.

6. The method of claim 4, wherein the exogenous cardiac glycoside comprises oleandrin, ouabain, digoxin, digitoxin, or bufalin.

7. A method for reducing population of tumor cells comprising:
treating the tumor cells to a radiosensitivity-enhancing amount of an exogenous cardiac glycoside to give treated tumor cells; and
exposing the treated tumor cells to an effective amount of ionizing radiation.

8. The method of claim 7, wherein the exogenous cardiac glycoside comprises oleandrin, ouabain, digoxin, digitoxin, or bufalin.

9. A method for reducing population of tumor cells comprising:
treating the tumor cells to a radiosensitivity-enhancing amount of an exogenous oleandrin to give treated tumor cells; and
exposing the treated tumor cells to an effective amount of ionizing radiation.
